(19) United States
(12) Patent Application Publication
(10) Pub. No.: US 2024/0298592 A1
(43) Pub. Date: Sep. 12, 2024

(54) USE OF HIGHER ALIPHATIC ALCOHOL IN IMPROVING NITROGEN FIXING CAPACITY AND DROUGHT RESISTANCE CAPACITY OF LEGUMES

(71) Applicant: Zhuhai Runnong Science and Technology Co. Ltd, Zhuhai (CN)

(72) Inventors: Dianxian Cai, Zhuhai (CN); Yunhao Sun, Zhuhai (CN); Xiaoming Peng, Zhuhai (CN); Guohui Yu, Zhuhai (CN); Di Qin, Zhuhai (CN)

(21) Appl. No.: 18/651,700

(22) Filed: May 1, 2024

Related U.S. Application Data
(63) Continuation of application No. PCT/CN2021/128777, filed on Nov. 4, 2021.

(51) Int. Cl. A01H 1/00; A01N 25/04; A01N 31/02; A01P 21/00

(52) U.S. Cl. CPC A01H 1/104; A01N 25/04; A01N 31/02; A01P 21/00

(57) ABSTRACT

The present invention provides a use of a higher aliphatic alcohol in preparing a preparation for increasing a content of lysophosphatidylcholine in legumes, and the higher aliphatic alcohol increases a quantity of nodules by increasing the content of lysophosphatidylcholine in the legumes, thereby improving a nitrogen fixing capacity of the legumes. The present invention also provides a use of a higher aliphatic alcohol in preparing a preparation for improving a drought resistance of legumes, and the preparation containing the higher aliphatic alcohol improves the drought resistance of the legumes by increasing a transcriptional level of genes related to the phenylpropanoid metabolic pathway, increasing a transcriptional level of genes related to the isoflavone biosynthetic pathway and increasing a content of an isoflavone compound in the legumes.

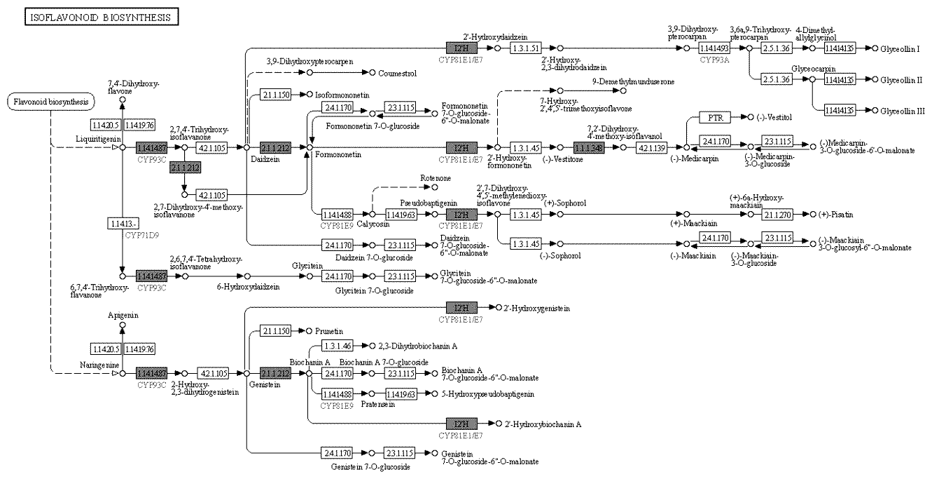

### CROSS-REFERENCE TO RELATED APPLICATIONS

The present application is a Continuation Application of PCT Application No. PCT/CN2021/128777, filed on Nov. 4, 2021, the entire contents of which are hereby incorporated by reference.

### TECHNICAL FIELD

The present invention relates to a use of higher aliphatic alcohol in improving nitrogen fixing capacity and drought resistance capacity of legumes.

### DESCRIPTION OF THE PRIOR ART

As an important economic crop, legumes are important sources of starch, protein, oil and vegetables in human food, and are also important feeding raw materials for animal husbandry and aquaculture. Among them, peanut oil and soybean oil are important plant-derived edible oils, and a soybean meal is also an important feed protein source. However, edible oil and feed protein in China depend on import for a long time. According to data shown by the General Administration of Customs, in 2016, a foreign dependence on edible oil in China reached 67.7%. Soybean imports in China in 2020 were 100.33 million tons, up 13.3% year-on-year. An import dependence of edible oil and soybean is too high, and is easy to be restricted by foreign market conditions, thereby posing a threat to grain and oil security in China.

Through a breeding of new varieties with resistance and high yield and an application of a green prevention and control technology for pests and diseases, a level of agricultural production of peanuts, soybeans and other leguminous crops in China has been advanced to a certain extent. However, an overall yield of soybeans and peanuts is low, frequent occurrences of diseases and insect pests, frequent bad weather, serious obstacles to continuous cropping, excessive use of chemical pesticides and fertilizers, and poor post-harvest quality and other key issues still seriously restrict a healthy and rapid development of related industries.

### SUMMARY OF THE DISCLOSURE

In order to overcome the shortcomings of prior art, a purpose of the present invention is to provide a use of higher aliphatic alcohol in improving nitrogen fixing capacity and drought resistance capacity of legumes.

In order to solve the above problems, the present invention provides the following technical solution.

The present invention provides a use of a higher aliphatic alcohol in preparing a preparation for increasing a content of lysophosphatidylcholine in legumes, and a use of increasing a quantity of nodules is achieved by increasing the content of lysophosphatidylcholine in the legumes through the higher aliphatic alcohol.

In one embodiment, a use of improving a nitrogen fixing capacity of the legumes is achieved by increasing the quantity of nodules of the legumes through the higher aliphatic alcohol.

On the other hand, the present invention also provides a use of a higher aliphatic alcohol in preparing a preparation for increasing a content of an isoflavone compound in legumes, wherein: the isoflavone compound is 3,9-dihydroxypterocarpan.

In one embodiment, a use of increasing a content of phytoalexins in the legumes is achieved by increasing a content of the 3,9-dihydroxypterocarpan in the legumes through the higher aliphatic alcohol.

On the other hand, the present invention also provides a use of a higher aliphatic alcohol in preparing a preparation for increasing a content of an isoflavone compound in legumes, and the isoflavone compound is calycosin and glycitein.

In one embodiment, a use of improving an anti-adversity activity of the legumes is achieved by increasing a content of the calycosin and glycitein in the legumes through the higher aliphatic alcohol.

On the other hand, the present invention also provides a use of a higher aliphatic alcohol in preparing a preparation for improving a germination rate and a germination potential, and shorting a germination time of peanuts.

The present invention provides a use of a higher aliphatic alcohol in preparing a preparation for improving a drought resistance of legumes, and a use of improving the drought resistance of the legumes is achieved through the preparation containing the higher aliphatic alcohol by increasing a transcriptional level of genes related to the phenylpropanoid metabolic pathway, increasing a transcriptional level of genes related to the isoflavone biosynthetic pathway and increasing a content of an isoflavone compound in the legumes.

In the present invention, the higher aliphatic alcohol is one of dodecanol and cetyl alcohol or a mixture of dodecanol and cetyl alcohol.

In one embodiment, the preparation is a water emulsion, and the water emulsion includes the higher aliphatic alcohol, an emulsifier, a thickener and water.

The preparation containing higher fatty alcohol of the present invention is not limited to the water emulsion, all dosage forms as long as adopt the inventive concept of the present invention, the dosage forms are applicable. For example, wettable powder, emulsion, sprayable solution, concentrated emulsion, aerosol and seed coating agent.

Compared with prior art, the beneficial effects of the present invention are as follows:

The present invention uses advanced transcriptome and metabolome sequencing and analysis technologies, to screen and determine that the water emulsion containing higher aliphatic alcohol of the present invention can affect the content of signal substances lysophosphatidylcholine and lysophosphatidylethanolamine in the legumes, the transcriptional levels of genes related to phenylpropanoid metabolism pathway and isoflavone biosynthesis pathway by an external application, and isoflavones are accumulated, thereby improving the nitrogen fixing capacity and drought resistance capacity of legumes.

The present invention will be clearly and completely described below, in conjunction with the drawing and the embodiment.

### BRIEF DESCRIPTION OF DRAWING

The drawing shows a schematic diagram of isoflavone biosynthesis pathway in peanut leaves according to an embodiment of the present invention.

### DESCRIPTION OF EMBODIMENTS

### Example 1

A water emulsion containing a higher aliphatic alcohol is provided in the embodiment, and the water emulsion consists of the following components in a weight percentage: 24% dodecanol, 3% cetyl alcohol, 3% emulsifier, 5% thickener, and the rest is water.

In the example, the emulsifier is aliphatic acid polyoxyethylene ester, and the thickener is methyl cellulose.

A method of preparing the water emulsion is also provided in the example, including the following steps:

Putting the dodecanol and the cetyl alcohol into a container and heating to 60 degrees to melt; then adding the emulsifier to the container, processing through a high shear homogenizer, and rotating for 10 minutes with a rotating speed of 5000 rotation per minute; then adding 60 degree water to the container, processing through the high shear homogenizer, and rotating for 10 minutes with a rotating speed of 10000 rotation per minute; cooling down to 40 degrees, and then adding the thickener to the container, processing by the high shear homogenizer, and rotating for 30 minutes with a rotating speed of 10000 rotation per minute, the water emulsion is obtained.

### Example 2

A water emulsion containing a higher aliphatic alcohol is provided in the example, and the water emulsion consists of the following components in a weight percentage: 24% dodecanol, 3% emulsifier, 5% thickener, and the rest is water.

In the example, the emulsifier is aliphatic acid polyoxyethylene ester, and the thickener is methyl cellulose.

A method of preparing the water emulsion is also provided in the example, including the following steps:

Putting the dodecanol into a container and heating to 60 degrees to melt; then adding the emulsifier to the container, processing through a high shear homogenizer, and rotating for 10 minutes with a rotating speed of 5000 rotation per minute; then adding 60 degree water to the container, processing through a high shear homogenizer, and rotating for 10 minutes with a rotating speed of 10000 rotation per minute; cooling down to 40 degrees, and then adding the thickener to the container, processing by the high shear homogenizer, and rotating for 30 minutes with a rotating speed of 10000 rotation per minute, the water emulsion is obtained.

### Example 3

A water emulsion containing a higher aliphatic alcohol is provided in the example, and the water emulsion consists of the following components in a weight percentage: 3% cetyl alcohol, 3% emulsifier, 5% thickener, and the rest is water.

In the example, the emulsifier is aliphatic acid polyoxyethylene ester, and the thickener is methyl cellulose.

A method of preparing the water emulsion is also provided in the example, including the following steps:

Putting the cetyl alcohol into a container and heating to 60 degrees to melt; then adding the emulsifier to the container, processing through a high shear homogenizer, and rotating for 10 minutes with a rotating speed of 5000 rotation per minute; then adding 60 degree water to the container, processing through the high shear homogenizer, and rotating for 10 minutes with a rotating speed of 10000 rotation per minute; cooling down to 40 degrees, and then adding the thickener to the container, processing by the high shear homogenizer, and rotating for 30 minutes with a rotating speed of 10000 rotation per minute, the water emulsion is obtained.

### Example 4

A water emulsion is provided in the example, and the water emulsion consists of the following components in a weight percentage: 3% emulsifier, 5% thickener, and the rest is water.

In the example, the emulsifier is aliphatic acid polyoxyethylene ester, and the thickener is methyl cellulose.

Putting the emulsifier to a container, processing through a high shear homogenizer, and rotating for 10 minutes with a rotating speed of 5000 rotation per minute; then adding 60 degree water to the container, processing through the high shear homogenizer, and rotating for 10 minutes with a rotating speed of 10000 rotation per minute; cooling down to 40 degrees, and then adding the thickener to the container, processing by the high shear homogenizer, and rotating for 30 minutes with a rotating speed of 10000 rotation per minute, the water emulsion is obtained.

Example 5: an effect of the water emulsion containing the higher aliphatic alcohol on contents of lysophosphatidylcholine and lysophosphatidylethanolamine in peanuts.

Lysophosphatidylcholine and lysophosphatidylethanolamine are important signal substances in plant cells, a normal physiological source is formed after phospholipids are hydrolyzed by phospholipase to remove a long carbon chain, which can be induced by various pressures. Lysophosphatidylcholine plays an important role in a root nodule formation. Lysophosphatidylethanolamine can activate an activity of phenylalanine ammonia-lyase, a key enzyme related to immunity and resistance to abiotic stress, and inhibit a plant premature senescence. Peanut samples are treated with the water emulsion containing a higher aliphatic alcohol of the present invention, a metabolite determination and a transcriptome sequencing are performed, and the effect of the water emulsion of the present invention on the content of lysophosphatidylcholine and lysophosphatidylethanolamine in peanuts is analyzed.

(1) Aliphatic alcohol sample: the water emulsion made by the present invention listed in Table 1.

TABLE 1
sample numbers

| | sample numbers | | | | |
|---|---|---|---|---|---|
| | sample | sample | sample | sample | sample |
| | S-compo- | A-compo- | B-compo- | C-compo- | CK |
| | nent | nent | nent | nent | (comparison) |
| raw | Example 1 | Example 2 | Example 3 | Example 4 | clean water |
| materials | | | | | |

(2) Test plants: peanut (Cultivated species Shanyou No. 35).

Culture conditions: 70/0 μmol m⁻² s⁻¹ (light/dark cycle light intensity), 14 hours/10 hours (time of light/dark cycle), 27° C./24° C., 70% relative humidity, four weeks old

(3) Sample processing and data collection.

Healthy plants with similar growth status in the group are selected, and the leaves are sprayed using the dilutions of the above samples (900 times diluted with water), until the leaves are completely covered with a liquid film. The comparison group is sprayed with a same amount of sterile water for diluting a stock solution. A second spraying treatment is performed after 48 hours. Three biological replicates are provided in each group.

72 hours after a first treatment, 4 grams of leaf samples are collected from each group. The leaf samples are quickly frozen in liquid nitrogen for 3 minutes, kept warm with dry ice and sent to a laboratory of Wuhan Metware Biotechnology Inc. for metabolomics sequencing (All metabolome and transcriptome sequencing data below are provided by Wuhan Metware Biotechnology Inc.).

(4) An effect of treatment on the content of two signal substances.

After peanut leaves are treated with a mixed preparation of dodecanol and cetyl alcohol (S), dodecanol preparation (A), emulsifier (C) and CK (clean water treatment) for 72 hours, detection results of the signal substances lysophosphatidylcholine (12:0 (12 carbons without double bonds)) and lysophosphatidylethanolamine (16:3 (16 carbons with three double bonds)) in physiological metabolites are shown in Table 2. The detection results of detecting an average number of three biological replicates show that, the mixed sample compared with the clean water comparison, a content of lysophosphatidylcholine is up-regulated 4070 times; and the mixed sample compared with an emulsifier treatment, the content of lysophosphatidylcholine is also up-regulated 4070 times, indicating that an increase of the signal substance is not caused by the emulsifier. A dodecanol separate treatment compared with the clean water comparison, the content of lysophosphatidylcholine is up-regulated 1850 times; and the dodecanol separate treatment compared with the emulsifier separate treatment, the content of lysophosphatidylcholine is up-regulated 1850 times, indicating that the up-regulation of signal substance caused by dodecanol treatment is also not caused by the emulsifier. The content of signal substance in the emulsifier treatment is consistent with the content of signal substance in the clean water treatment.

Similarly, the mixed sample compared with the clean water comparison, a content of another signal substance lysophosphatidylethanolamine is up-regulated 960 times, being consistent with an increase multiple compared with the emulsifier treatment, indicating that the up-regulation of the content of the signal substance is caused by a treatment with higher aliphatic alcohols. The increase multiple of the dodecanol separate treatment compared with the comparison and compared with the emulsifier treatment are consistent, and lower than that of the mixed treatment of dodecanol and cetyl alcohol, which is consistent with a situation of lysophosphatidylcholine.

The content change results of the two signal substances caused by the separate treatment and mixed treatment of higher aliphatic alcohols, showing that the content of the above two signal substances can be up-regulated by both separate treatment and mixed treatment of higher aliphatic alcohols.

TABLE 2
a content ratio of lysophosphatidylcholine and
lysophosphatidylethanolamine in peanut plant leaves
in each treatment group (increase multiple)

| | peanut leaves | peanut leaves |
|---|---|---|
| | lysophosphatidylcholine | lysophosphatidylethanolamine |
| | (12:0) | (16:3) |
| S/comparison | 4070 | 960 |
| S/C | 4070 | 960 |
| A/comparison | 1850 | 420 |
| A/C | 1850 | 420 |
| C/comparison | 1 | 1 |

Labeling of types of lysophospholipids: 12:0 means 12 carbons with no double bonds, 16:3 means 16 carbons with three double bonds.

Example 6: an effect of the water emulsion containing the higher aliphatic alcohol on transcriptional levels of genes related to phenylpropanoid metabolism pathway in peanut.

Lysophospholipids are involved in a regulation of phenylalanine ammonia lyase activity, which is a specific metabolic pathway for signal substances, and is also one of the most important plant secondary metabolic pathways, thereby playing an important role in the plant growth and development and plant-environment interactions. The pathway includes a plurality of branch pathways, to produce isoflavone metabolites. The isoflavone metabolites are involved in helping plant cells reduce ultraviolet damage, resist disease occurrence, and tolerate uncomfortable temperatures and high-salt drought conditions and other physiological processes. The content of lysophospholipid signal substances in the peanut leaves can be significantly up-regulated by the higher aliphatic alcohol preparations of the present invention, indicating that the higher aliphatic alcohol preparations of the present invention may further affect downstream metabolic pathways. Therefore, transcriptional levels of key enzyme coding genes of phenylpropanoid metabolic pathway is further detected in the peanut leaves treated with S sample, A sample, B sample and clean water.

(1) Aliphatic alcohol sample: the same as Example 5.

(2) Test plants: the same as Example 5.

(3) Sample processing and data collection: the same as Example 5, and a transcriptome sequencing is completed by Wuhan Metware Biotechnology Inc.

(4) Treating an effect on transcriptional levels of genes related to the phenylpropanoid metabolic pathway.

Effects of different treatments on the transcriptional levels of genes related to the phenylpropanoid metabolism pathway in the peanut leaves are shown in Table 3. Average results of three biological replicates of transcriptome determination show that whether the dodecanol separate treatment, the cetyl alcohol separate treatment, or the mixed treatment of dodecanol and cetyl alcohol, the transcriptional levels of some important genes can be significantly up-regulated. Overall, the mixed treatment of dodecanol and cetyl alcohol is better than the dodecanol separate treatment and the cetyl alcohol separate treatment in regulating gene transcriptional levels, being consistent with the content results of signal substance measured by a metabolome, and showing that the up-regulation of the transcriptional levels of genes related to the phenylpropanoid metabolism pathway in the peanut leaves can be caused by the separate treatment of dodecanol or cetyl alcohol, or the mixed treatment of dodecanol and cetyl alcohol.

TABLE 3
a difference ratio of the transcriptional levels of genes related
to phenylpropanoid metabolism pathway in peanut plant leaves.

| | peanut | peanut | peanut |
|---|---|---|---|
| | A/compar- | B/compar- | S/compar- |
| gene encoding protein name | ison | ison | ison |
| scopoletin glucosyltransferase | 4.17 | 3.55 | 3.40 |
| caffeoyl-CoA O-methyltransferase | 2.01 | 2.22 | 14.62 |
| coniferyl-aldehyde dehydrogenase | 3.69 | 2.86 | 2.39 |
| cinnamoyl-CoA reductase | 12.5 | 11.52 | 3.58 |
| cinnamyl-alcohol dehydrogenase | 2.04 | 2.38 | 7.22 |
| Peroxidase | 2.24 | 3.61 | 9.53 |
| phenylalanine ammonia-lyase | 2.36 | <2.00 | 5.77 |
| caffeic acid 3-O-methyltransferase | <2.00 | 8.10 | 4.42 |
| chalcone isomerase | 2.28 | 2.25 | 3.81 |
| chalcone synthase | <2.00 | 2.11 | 9.31 |
| isoflavone 3′-hydroxylase | 2.13 | 2.37 | 2.25 |

Example 7: an effect of the water emulsion containing the higher aliphatic alcohol on a gene transcription and a metabolite content related to a biosynthetic pathway of peanut isoflavones.

The phenylpropanoid metabolic pathway is involved in numerous physiological activities of plants, in order to further verify that the preparation of the present invention can regulate the gene transcriptional level and the metabolite content of plant phenylpropanoid metabolism-related pathways, the biosynthetic pathway of isoflavone is selected as a target, the peanut leaves are treated with S sample and clean water for 72 hours, the gene transcriptional levels related to pathway and corresponding metabolites are measured, and a joint analysis is performed. The results are shown in the drawing and Tables 4 and 5.

(1) Aliphatic alcohol sample: the same as Example 5.

(2) Test plants: the same as Example 5.

(3) Sample processing and data collection: the same as Example 5, and a determination of transcriptome and metabolome is completed by Wuhan Metware Biotechnology Inc.

(4) Treating an effect on the gene transcription and metabolite content related to the biosynthetic pathway of isoflavone.

The biosynthetic pathway of isoflavones in peanut leaves is shown in the drawing, among them, the transcriptional levels of a plurality of genes are up-regulated after the treatment with the mixed preparation of dodecanol and cetyl alcohol, gray represents that the corresponding gene (rectangular) transcriptional level or metabolite (circle) content after S preparation treatment is significantly up-regulated compared with the comparison group. The up-regulation levels in the average of three biological replicates of related genes are shown in Table 4, among them, a gene transcriptional level of 2-hydroxyisoflavanone synthase is up-regulated 5.80 times, a gene transcriptional level of isoflavone 4′-O-methyltransferase is up-regulated 5.80 times, a gene transcriptional level of isoflavone/4′-methoxyisoflavone 2′-hydroxylase is up-regulated 2.25 times, and a gene transcriptional level of vestitone reductase is up-regulated 4.21 times. Correspondingly, the contents of several important isoflavones are also up-regulated significantly, among them, a content of 3,9-dihydroxypterocarpan is up-regulated 1223.67 times, a content of calycosin is up-regulated 2657.76 times, and a content of glycitein is up-regulated 1982.35 times. Research shows that 3,9-dihydroxypterocarpan is an important phytoalexin precursor and can improve crop resistance to pests and diseases; and calycosin and glycitein have been shown to have significant anti-adversity activity.

TABLE 4
a difference ratio of transcriptional levels of genes related to biosynthetic
pathway of isoflavone in the peanut plant leaves (S/comparison)

| | labeled in the drawing | | | |
|---|---|---|---|---|
| | 1.14.14.87 | 2.1.1.212 | 1.14.14.90 | 1.1.1.348 |
| coding protein | 2- | isoflavone 4′-o- | isoflavone/4′- | vestitone |
| | hydroxyisoflavanone | methyltransferase | methoxyisoflavone | reductase |
| | synthase | | 2′-hydroxylase | |
| increase multiple | 5.80 | 4.53 | 2.25 | 4.21 |

TABLE 5
a difference ratio of marked metabolite content of biosynthetic pathway
of isoflavone in the peanut plant leaves (S/comparison)

| labeled in | | |
|---|---|---|
| the | | formononetin |
| drawing | formononetin | 7-o-glucoside |
| substance | c₁₆h₁₂o₄ | c₂₂h₂₂o₉ |
| | 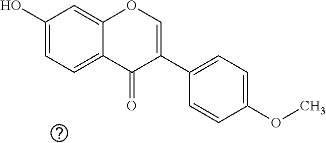 [SMILES: COc1ccc(-c2coc3cc(O)ccc3c2=O)cc1] | 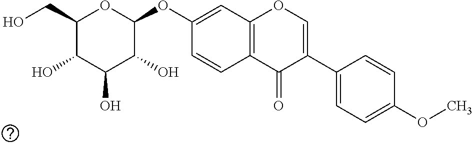 [SMILES: COc1ccc(-c2coc3cc(O[C@@H]4O[C@H](CO)[C@@H](O)[C@H](O)[C@H]4O)ccc3c2=O)cc1] |
| increase | 2.98 | 2.54 |
| multiple | | |
| labeled in | | |
| the | | |
| drawing | 3,9-dihydroxypterocarpan | calycosin |
| substance | c₁₅h₁₂o₄ | c₁₆h₁₂o₅ |
| | 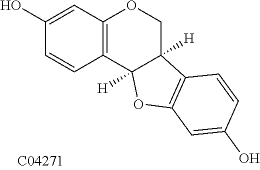 [SMILES: Oc1ccc2c(c1)O[C@H]1c3ccc(O)cc3OC[C@@H]21] | 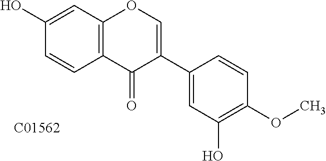 [SMILES: COc1ccc(-c2coc3cc(O)ccc3c2=O)cc1O] |
| increase | 1223.67 | 2657.76 |
| multiple | | |
| labeled in | | |
| the | | |
| drawing | glycitein | pratensein |
| substance | c₁₆h₁₂o₅ | c₁₆h₁₂o₆ |
| | 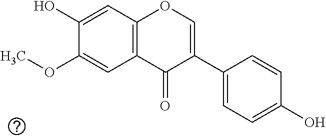 [SMILES: COc1cc2c(=O)c(-c3ccc(O)cc3)coc2cc1O] | 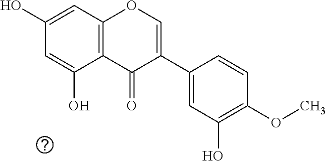 [SMILES: COc1ccc(-c2coc3cc(O)cc(O)c3c2=O)cc1O] |
| increase | 1982.35 | 2.16 |
| multiple | | |

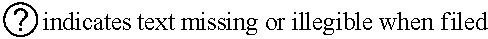

Example 8: an effect of the water emulsion containing the higher aliphatic alcohol on a content of soybean lysophosphatidylethanolamine.

Based on the research of Examples 5, 6 and 7, soybean plant samples are treated with the water emulsion containing a higher aliphatic alcohol and clean water of the present invention, the transcriptome and the metabolome are measured, and the effect of the water emulsion containing a higher aliphatic alcohol of the present invention on soybean plant physiology is analyzed.

(1) Aliphatic alcohol sample: the same as Example 5.

(2) Test plants: soybeans (HuaChun No. 1).

Culture conditions for each plant: 80/0 μmol m⁻² s⁻¹ (light/dark cycle light intensity), 14 hours/10 hours (light/dark time cycle), 28° C./25° C., 70% relative humidity, four weeks old.

(3) Sample processing:

Healthy plants with similar growth status in the group are selected, and the leaves are sprayed using the dilutions of the above samples (900 times diluted with water), until the leaves are completely covered with a liquid film. The comparison group is sprayed with the same amount of sterile water for diluting a stock solution. A second spraying treatment is performed after 48 hours. Three biological replicates are set in each group. 72 hours after the first treatment, 4 grams of leaf samples are collected from each group. The leaf samples are quickly frozen in liquid nitrogen for 3 minutes, stored in dry ice and sent to the laboratory of Wuhan Metware Biotechnology Inc. to perform transcriptome and metabolome measurements.

(4) Effect of treatment on the content of lysophosphatidylethanolamine in soybean leaves.

After the soybean leaves are treated with S sample and clean water for 72 hours, an average number of three biological replicates is measured, and finding that the content of the signal substance lysophosphatidylethanolamine (24:0 (24 carbons without double bonds)) in soybean leaves treated with S sample is 2.44 times higher than that in soybean leaves treated with clean water (Table 6).

TABLE 6
a content ratio of lysophosphatidylethanolamine in soybean
plant leaves in each treatment group (increase multiple)

| | soybean leaves |
|---|---|
| | lysophosphatidylethanolamine |
| | (24:0) |
| S/comparison | 2.44 |

Labeling of types of lysophospholipids: 24:0:0 means 24 carbons with no double bonds.

Example 9: an effect of the water emulsion containing the higher aliphatic alcohol on transcriptional levels of genes related to phenylpropanoid metabolism pathway in soybeans.

Phenylpropanoid metabolism is one of the most important plant secondary metabolic pathways, and plays an important role in the plant growth and development and plant-environment interactions. The pathway includes a plurality of branch pathways, producing metabolites such as lignin, isoflavones and the like. In soybeans, isoflavones attract rhizobium and induce an expression of nod gene, thereby inducing a formation of root nodules. (Journal of Northeast Agricultural University, October 2007, [symbol] The pathway of isoflavone biosynthesis and its regulation [symbol] Author: M A Junlan, L I Cheng, etc.).

Lignin is mainly accumulated in secondary cell walls and participates in a process of providing mechanical support, water transport, resistance to pests and diseases, and resistance to non-physiological stress. The isoflavone metabolites assist plant cells in reducing ultraviolet damage, removing reactive oxygen species, resisting an occurrence of diseases, and tolerating uncomfortable temperatures and high-salt drought conditions and other physiological processes. Isoflavones are also an important factor in soybean quality

(1) Aliphatic alcohol sample: the same as Example 5.

(2) Test plants: the same as Example 8.

(3) Sample processing and data collection:the same as Example 8.

(4) Treating an effect on the transcriptional levels of genes related to the phenylpropanoid metabolic pathway in soybean leaves.

After the soybean leaves are treated with S sample and clean water for 72 hours, a transcriptome differential analysis is performed, and the results of measuring the average number of three biological replicates are shown in Tables 7 and 8. The phenylpropanoid metabolic pathway and the transcriptional levels of key enzyme coding genes of isoflavone biosynthesis pathway changed significantly, and are up-regulated significantly in the S-treated samples.

TABLE 7
a difference ratio of the transcriptional levels of genes
related to phenylpropanoid metabolic pathway in the soybean
plants leaves in each treatment group (increase multiple)

| | soybean |
|---|---|
| gene encoding protein name | S/comparison |
| scopoletin glucosyltransferase | 2.68 |
| shikimate O-hydroxycinnamoyltransferase | 4.46 |
| cinnamyl-alcohol dehydrogenase | 3.90 |
| Peroxidase | 5.31 |
| caffeic acid 3-O-methyltransferase | 2.15 |
| ferulate-5-hydroxylase | 2.55 |
| beta-glucosidase | 3.11 |
| chalcone synthase | 2.03 |
| flavonol synthase | 2.36 |
| flavonoid 3′-monooxygenase | 7.41 |
| anthocyanidin reductase | 3.68 |
| isoflavone-7-O-methyltransferase | 2.32 |
| isoflavone 3′-hydroxylase | 5.59 |
| phenolic glucoside malonyltransferase | 2.85 |
| Arylacetamide deacetylase | 2.96 |
| isoflavone 7-O-glucosyltransferase | 2.08 |
| 7-ethoxycoumarin O-deethylase | 3.45 |

TABLE 8
a difference ratio of the transcriptional levels of genes
related to isoflavone biosynthetic pathway in the soybean
plants leaves in each treatment group (increase multiple)

| | soybean |
|---|---|
| gene encoding protein name | S/comparison |
| 2.hydroxyisoflavanone dehydratase | 2.97 |
| isoflavone-7-O-methyltransferase | 2.33 |
| isoflavone 7-O-glucosyltransferase | 2.08 |
| isoflavone 7-O-glucoside-6″-O-malonyltransferase | 2.85 |
| isoflavone/4′-methoxyisoflavone 2′-hydroxylase | 5.60 |
| 3,9-dihydroxypterocarpan 6a-monooxygenase | 3.46 |

Example 10: an effect of the water emulsion containing the higher aliphatic alcohol on physiological phenotypes of peanuts.

Through Examples 5, 6, and 7, finding that the water emulsion containing a higher aliphatic alcohol can significantly affect the signal substances lysophosphatidylcholine and lysophosphatidylethanolamine of peanuts, and further affect the phenylpropane metabolic pathway and the biosynthesis of isoflavones. The specific physiological phenotypes of the physiological pathways include disease resistance, yield increase, ultraviolet rays resistance and the like. In order to verify that the physiological metabolism pathway caused by the higher aliphatic alcohols of the present invention indeed has the above-mentioned physiological activity, a verification under controllable conditions of environmental variables is carried out in a laboratory.

(1) Test plants: the same as Example 5.

(2) Test method:

Dressing peanut seeds with 150 times diluted S sample, A sample, B sample and clean water respectively, drying, and then sowing; determining a germination potential, a germination rate, a germination index, an average germination day and a vigor index; and spraying the leaves (900 times dilution), then treating the roots with 5% polyethylene glycol 6000 and growing under simulated drought conditions for 8 days, and then determining a plant height, a proline content of leaf tissue, a malondialdehyde (MDA) content, a peroxidase (POD) activity, and a plant dry weight physiological and biochemical indicator.

(3) Treating an effect on peanut physiology:

Results of measuring the average number of three biological replicates are shown in Tables 9 and 10. The results show that the mixture of dodecanol and cetyl alcohol can significantly improve the germination potential of peanuts, which is higher than the germination potential of the separate treatment of dodecanol or cetyl alcohol, and the comparison; and the germination potential of the separate treatment of dodecanol or cetyl alcohol is also significantly higher than the germination potential of the comparison, however, there is no difference between the two aliphatic alcohols treated separately. The germination rate, the germination index and the vigor index shows the same rule. The mixed treatment and the separate treatment of dodecanol can significantly shorten an average germination day, and the cetyl alcohol treatment can also significantly shorten the germination day compared with the comparison. It shows that the mixture of dodecanol and cetyl alcohol has obvious phenotypes in improving a peanut germination rate and a bud vigor and shortening a germination time. Among them, a, b and c represent a significant difference relationship of data in statistics. Group b is more significant than group c, and group a is more significant than group b.

TABLE 9
physiological indicators of peanut seed germination after treatment

| | germination | germination | germination | average germination | |
|---|---|---|---|---|---|
| name | potential | rate | index | day | vigor index |
| comparison | 81 ± 2.3% c | 82 ± 0.1% c | 16.34 ± 0.21c | 4.89 ± 0.78a | 224.12 ± 2.31c |
| A | 84 ± 1.2% b | 85 ± 0.4% b | 21.22 ± 0.34b | 4.33 ± 0.21c | 234.45 ± 1.25b |
| treatment | | | | | |
| B treatment | 84 ± 2.1% b | 86 ± 0.8% b | 23.82 ± 0.44b | 4.40 ± 0.15b | 238.12 ± 3.12b |
| S treatment | 85 ± 2.4% a | 87 ± 1.1% a | 24.75 ± 0.15a | 4.12 ± 0.21c | 244.34 ± 3.21a |

Phenylpropanoid metabolic pathway and isoflavone metabolites can increase plant resistance to adversity, through a simulated drought experiment, determining that the separate or mixed treatment of dodecanol and cetyl alcohol can significantly improve the resistance of peanut to drought, and the plant height of peanuts is significantly up-regulated by the separate or mixed treatment of dodecanol and cetyl alcohol. The proline content of leaves treated with the mixture of dodecanol and cetyl alcohol is significantly higher than the proline content of leaves treated with dodecanol or cetyl alcohol separately and the comparison, and the proline content of leaves treated with dodecanol or cetyl alcohol separately is also significantly higher than the proline content of leaves of the comparison. A regularity of malondialdehyde content is consistent with a regularity of proline content. The POD content of mixed treatment is significantly higher than the POD content of other treatments and the comparison, and the separate treatment of dodecanol or cetyl alcohol is higher than the comparison but the difference is not significant. A data of the dry weight is consistent with the POD.

The results of physiological determination further verified that the higher aliphatic alcohols can indeed improve the resistance of peanut to drought by affecting the content of lysophosphatidylcholine and lysophosphatidylethanolamine signal substances.

TABLE 10
physiological indexes of peanut plants after simulated drought treatment

| | plant | proline | MDA | POD activity | dry weight |
|---|---|---|---|---|---|
| name | height(cm) | (μg/g) | (μmol/g) | (μ/mg) | (g) |
| comparison | 8.92 ± 0.41b | 18.21 ± 0.03c | 0.079 ± 0.02c | 21.89 ± 1.12b | 33.12 ± 2.12b |
| A treatment | 12.11 ± 1.21a | 27.73 ± 0.42b | 0.122 ± 0.01b | 25.12 ± 0.21a | 41.45 ± 2.55a |
| B treatment | 10.65 ± 0.31a | 26.73 ± 0.82b | 0.102 ± 0.04b | 26.30 ± 1.15a | 43.23 ± 3.12a |
| S treatment | 12.47 ± 2.1a | 28.11 ± 1.21a | 0.144 ± 0.02a | 26.68 ± 1.22a | 48.41 ± 4.11a |

Example 11: an effect of using the water emulsion containing the higher aliphatic alcohol in peanut field production

Sampling: Sample S is the water emulsion prepared by Example 1.

Crop: Peanut Shan You No. 35.

Location: Luoba Town, Shixing County, Shaoguan City, Guangdong Province.

Method: Selecting a 10-mu test group and a 10-mu comparison group at a production base to produce according to Table 11. mu is an area unit, and 1 mu equals about 0.067 hectares.

TABLE 11
a test scheme of peanut

| serial | processing | | |
|---|---|---|---|
| number | period | test group | comparison group |
| 1 | seed | Dilute the sample S 50 | |
| | dressing | times with water and | |
| | | mix the seed, dry and | |
| | | then sow the seed. | |
| 2 | seedling | After the 5-leaf stage, | At the same time |
| | stage | dilute sample S 900 | as the test group |
| | | times with water and | is treated, the |
| | | spray once. | same amount of |
| | | Sample dosage: 15 mL/mu, | clean water is |
| | | time | sprayed. |
| 3 | flowering | Dilute the sample S 900 | At the same time |
| | period | times with water and | as the test group |
| | | spray twice with an | is treated, the |
| | | interval of 10 days. | same amount of |
| | | Sample dosage: 15 mL/mu, | clean water is |
| | | time | sprayed. |
| 4 | podding | Dilute the sample S 900 | At the same time |
| | stage | times with water and | as the test group |
| | | spray once. | is treated, the |
| | | Sample dosage: 15 mL/mu, | same amount of |
| | | time | clean water is |
| | | | sprayed. |
| 5 | mature | Dilute the sample S 900 | At the same time |
| | stage | times with water and | as the test group |
| | | spray once. | is treated, the |
| | | Sample dosage: 15 mL/mu, | same amount of |
| | | time | clean water is |
| | | | sprayed. |

A comparison of the use effects is shown in Table 12.

TABLE 12
test results of peanut:

| serial | | | |
|---|---|---|---|
| number | item | test group | comparison group |
| 1 | growth status | germination rate: | germination rate: |
| | during | 98.6% | 91.1% |
| | germination | root length on the | root length on the |
| | period | 12th days: 4.5 cm | 12th days: 2.0 cm |
| 2 | growth status | plants are strong, | plants are normal, |
| | during seedling | leaves are green. | leaves are green. |
| | stage | | |
| 3 | growth status | plant height: 51.2 cm | plant height: 42.5 cm |
| | during flow- | number of bracts per | number of bracts per |
| | ering period | plant: 35.4 | plant: 18.7 |
| 4 | fruit harvest | fruit weight per | fruit weight per |
| | situation | plant: 83.2 g | plant: 71.5 g |
| | | a proportion of | a proportion of |
| | | rotten fruit: 0.9% | rotten fruit: 11.2% |
| 5 | root | roots are many and | roots are few and |
| | condition | long, and nodules | short, nodules are |
| | | are large and dense, | small and sparse, |
| | | 530 per plant | 210per plant |

Throughout the peanut growth cycle, the germination rate, the growth status, the number of bracts and the number of nodules of the peanuts in the test group treated with the water emulsion containing a higher aliphatic alcohol of the present invention are better than that in the comparison group, and no pest damage and plant disease occurred. From the final fruit harvest situation, the rotten fruit rate of the test group is less than 1%, and the peanut weight per plant of the test group is up-regulated by 16.36% compared with the comparison group.

Example 12: use effect of the water emulsion containing higher aliphatic alcohol in soybean field production.

Sampling: Sample S is the water emulsion prepared by Example 1.

Crop: Soybean HuaChun No. 1.

Location: Zhangshi Town, Qujiang District, Shaoguan City, Guangdong Province.

Method: Selecting a 10-mu test group and a 10-mu comparison group in a production base to produce according to Table 13.

TABLE 13
soybean test scheme

| serial | processing | | |
|---|---|---|---|
| number | period | test group | comparison group |
| 1 | seed | Dilute the sample S 50 times | |
| | dressing | with water and mix the seed, | |
| | | dry and then sow the seed. | |
| 2 | seedling | After a 7-leaf stage, dilute | At the same time as |
| | stage | sample S 900 times with | the test group is |
| | | water and spray once. | treated, the same |
| | | Sample dosage: 15 mL/mu, | amount of clean |
| | | time | water is sprayed. |
| 3 | flowering | Dilute the sample S 900 times | At the same time as |
| | period | with water and spray twice | the test group is |
| | | with an interval of 10 days. | treated, the same |
| | | Sample dosage: 15 mL/mu, | amount of clean |
| | | time | water is sprayed. |
| 4 | podding | Dilute the sample S 900 times | At the same time as |
| | stage | with water and spray once. | the test group is |
| | | Sample dosage: 15 mL/mu, | treated, the same |
| | | time | amount of clean |
| | | | water is sprayed. |

Throughout the soybean growth cycle, the growth status, the number of pods, the number of nodules of the soybean in the test group treated with the water emulsion containing a higher aliphatic alcohol of the present invention are better than that in the comparison group.

During the test, both soybeans in the test group and the comparison group are suffered more than 40 days of high temperature and drought weather, and the comparison group is significantly affected.

A comparison of the use effects is shown in Table 14.

TABLE 14
soybeans test results:

serial

| number | item | test group | | comparison group | |
|---|---|---|---|---|---|
| 1 | average plant | 73.1 | cm | 45.3 | cm |
| | height | | | | |
| 2 | number of | 83.5 | | 28.2 | |
| | pods per plant | | | | |
| 3 | total weight | 59.8 | g | 20.0 | g |

| | | | |
|---|---|---|---|
| | of fruits | | |
| | per plant | | |
| 4 | root | Roots are many and | Roots are few and |
| | condition | long, and nodules are | short, nodules are |
| | | large and dense, | small and sparse, |
| | | 31 per plant. | 16 per plant. |

The comparison group is weak in growth due to more than 40 days of high temperature and drought weather, and it can be seen from the number of pods per plant and the total weight of fruits per plant that the comparison group has been in a state of harvest failure. The test group maintained normal growth conditions due to the effect of using the water emulsion containing a higher aliphatic alcohol of the present invention, and it can be seen from the final fruit harvest that the weight of soybeans per plant of the test group is up-regulated by 199.0% compared with the comparison group.

It can be seen from Examples 5, 6, 7, 8 and 9 that the treatment of legumes (peanuts, soybeans) with the water emulsion containing a higher aliphatic alcohol diluted with water can significantly affect the content of signal substances lysophosphatidylcholine and lysophosphatidylethanolamine in legumes, the transcriptional level of genes related to phenylpropanoid metabolism pathway and isoflavone biosynthesis pathway, and accumulate isoflavone substances; thereby improving the nitrogen fixing capacity, the drought resistance, the quality and the yield of legumes. It can be seen from Example 10 that the physiological phenotype test under the condition of controllable environmental variables in the laboratory proves that the seed germination, the seedling growth and the stress resistance index of legumes can be significantly affected by the water emulsion containing a higher aliphatic alcohol. The field application effect of Example 11 shows that the use of the water emulsion containing a higher aliphatic alcohol can increase the number of peanut nodules, reduce the incidence of bad fruit caused by diseases, improve the quality and increase the yield. The field application effect of Example 12 shows that the water emulsion containing higher aliphatic alcohol can increase the number of soybean nodules, improve the drought resistance of soybean and increase the yield.

It can be seen from Example 5 to Example 12, applying the water emulsion containing a higher aliphatic alcohol of the present invention to legumes, the content of the signal substances lysophosphatidylcholine and lysophosphatidylethanolamine in the legumes can be affected by an external application, thereby increasing the root nodule content of legumes to promote nitrogen fixing and increase yields. At the same time, the transcriptional levels of genes related to the phenylpropanoid metabolism pathway and the isoflavone biosynthesis pathway are up-regulated, and the isoflavones are accumulated to promote the drought resistance and improve the quality.

The above are merely some embodiments of the present invention, cannot be construed to limit the present invention. Any changes, modifications, alternatives and variations can be made in the embodiments without departing from the scope of the present disclosure, but these all fall into the protection scope of the present invention.

1. Use of a higher aliphatic alcohol in preparing a preparation for increasing a content of lysophosphatidylcholine in legumes, wherein: a use of increasing a quantity of nodules is achieved by increasing the content of lysophosphatidylcholine in the legumes through the higher aliphatic alcohol; and the higher aliphatic alcohol is one of dodecanol and cetyl alcohol or a mixture of dodecanol and cetyl alcohol.

2. The use of claim 1, wherein a use of improving a nitrogen fixing capacity of the legumes is achieved by increasing the quantity of nodules of the legumes through the higher aliphatic alcohol.

3. The use of claim 1, wherein the preparation is a water emulsion, and the water emulsion comprises the higher aliphatic alcohol, an emulsifier, a thickener and water.

4. Use of a higher aliphatic alcohol in preparing a preparation for increasing a content of an isoflavone compound in legumes, wherein: the t isoflavone compound is 3,9-dihydroxypterocarpan; and the higher aliphatic alcohol is one of dodecanol and cetyl alcohol or a mixture of dodecanol and cetyl alcohol.

5. The use of claim 4, wherein a use of increasing a content of phytoalexins in the legumes is achieved by increasing a content of the 3,9-dihydroxypterocarpan in the legumes through the higher aliphatic alcohol.

6. The use of claim 4, wherein the preparation is a water emulsion, and the water emulsion comprises the higher aliphatic alcohol, an emulsifier, a thickener and water.

7. Use of a higher aliphatic alcohol in preparing a preparation for increasing a content of an isoflavone compound in legumes, wherein: the isoflavone compound is calycosin and glycitein; and the higher aliphatic alcohol is one of dodecanol and cetyl alcohol or a mixture of dodecanol and cetyl alcohol.

8. The use of claim 7, wherein a use of improving an anti-adversity activity of the legumes is achieved by increasing a content of the calycosin and glycitein in the legumes through the higher aliphatic alcohol.

9. The use of claim 7, wherein the preparation is a water emulsion, and the water emulsion comprises the higher aliphatic alcohol, an emulsifier, a thickener and water.

10. Use of a higher aliphatic alcohol in preparing a preparation for improving a germination rate and a germination potential, and shorting a germination time of peanuts, wherein the higher aliphatic alcohol is one of dodecanol and cetyl alcohol or a mixture of dodecanol and cetyl alcohol.

11. The use of claim 10, wherein the preparation is a water emulsion, and the water emulsion comprises the higher aliphatic alcohol, an emulsifier, a thickener and water.

12. Use of a higher aliphatic alcohol in preparing a preparation for improving a drought resistance of legumes, wherein a use of improving the drought resistance of the legumes is achieved through the preparation containing the higher aliphatic alcohol by increasing a transcriptional level of genes related to the phenylpropanoid metabolic pathway, increasing a transcriptional level of genes related to the isoflavone biosynthetic pathway and increasing a content of an isoflavone compound in the legumes; wherein the higher aliphatic alcohol is one of dodecanol and cetyl alcohol or a mixture of dodecanol and cetyl alcohol.

13. The use of claim 12, wherein the preparation is a water emulsion, and the water emulsion comprises the higher aliphatic alcohol, an emulsifier, a thickener and water.
